(19) United States
(12) Patent Application Publication
(10) Pub. No.: US 2026/0174757 A1
(43) Pub. Date: Jun. 25, 2026

(54) COMPOSITIONS AND METHODS FOR TREATING PULMONARY ARTERIAL HYPERTENSION

(71) Applicant: Inhibikase Therapeutics, Inc., Wilmington, DE (US)

(72) Inventor: Milton H. Werner, Marietta, GA (US)

(21) Appl. No.: 19/542,047

(22) Filed: Feb. 17, 2026

Related U.S. Application Data
(63) Continuation of application No. PCT/US25/22429, filed on Apr. 1, 2025.
(60) Provisional application No. 63/573,253, filed on Apr. 2, 2024.

(51) Int. Cl. A61K 31/506

(52) U.S. Cl. CPC A61K 31/506

(57) ABSTRACT

The present disclosure provides compositions and methods for the prevention or treatment of pulmonary arterial hypertension.

### RELATED APPLICATIONS

This application claims the benefit of priority to U.S. Provisional Application No. 63/573,253, filed Apr. 2, 2024, the contents of which are incorporated herein by reference in their entirety.

### BACKGROUND

Pulmonary arterial hypertension (PAH) is an incurable and often fatal disease characterized by sustained elevation of pulmonary artery pressure and progressive vascular remodeling. Despite the approval of several therapies treating PAH, there is currently no cure and patients continue to have a poor prognosis with a 5-year survival rate of 57%. Accordingly, improved agents for treating PAH are needed.

### SUMMARY

In certain aspects, disclosed are methods of treating pulmonary arterial hypertension, comprising administering to a subject a compound of formula (I):

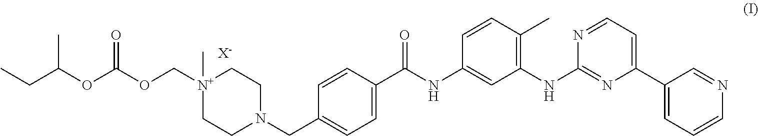

wherein X⁻ is a pharmaceutically acceptable anion.

In other aspects, disclosed are pharmaceutical composition comprising about 10 mg to about 1000 mg of a compound of formula (I):

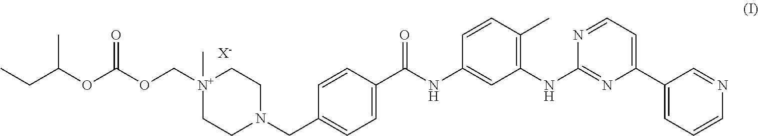

wherein X⁻ is a pharmaceutically acceptable anion;
and a pharmaceutically acceptable carrier or excipient; and uses thereof.

### BRIEF DESCRIPTION OF THE DRAWINGS

FIG. 1 shows the dose linearity of imatinib delivery by the mesylate salt of the compound of formula (I).

FIG. 2 shows concentration v. time data for the pivotal cohorts for determining doses of the mesylate salt of the compound of formula (I) equivalent to 400 mg or 600 mg imatinib mesylate.

### DETAILED DESCRIPTION

In certain aspects, disclosed are methods of treating pulmonary arterial hypertension, comprising administering to a subject a compound of formula (I):

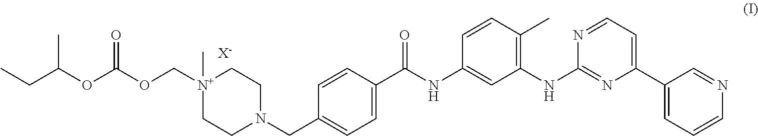

wherein X⁻ is a pharmaceutically acceptable anion.

In certain especially preferred embodiments, X⁻ is mesylate. In certain preferred embodiments, the subject is a human. In certain preferred embodiments, the method comprises administering the mesylate salt of the compound at a dose of about 10 mg to about 1000 mg. In some preferred embodiments, the dose of the mesylate salt of the compound is about 100 mg per day, about 200 mg per day, about 300 mg per day, about 400 mg per day, about 450 mg per day, about 500 mg per day, about 600 mg per day, about 700 mg per day, or about 800 mg per day. In some embodiments, the administration is repeated, preferably periodic (e.g., daily). In certain preferred embodiments, the compound is administered orally. In some especially preferred embodiments, the mesylate salt of the compound is administered at a dose of 300 mg per day. In other especially preferred embodiments, the mesylate salt of the compound of formula (I) is administered at a dose of 450 mg per day. In yet other especially preferred embodiments, the mesylate salt of the compound is administered at a dose of 500 mg per day. In some embodiments, the compound of formula (I) is administered at a dose equivalent to about 10 mg of the mesylate salt of the compound of formula (I) to about 1000 mg of the mesylate salt of the compound of formula (I). In certain embodiments, the compound of formula (I) is administered at a dose equivalent to about 100 mg of the mesylate salt of the compound of formula (I), about 200 mg of the mesylate salt of the compound of formula (I), about 300 mg of the mesylate salt of the compound of formula (I), about 400 mg of the mesylate salt of the compound of formula (I), about 450 mg of the mesylate salt of the compound of formula (I), about 500 mg of the mesylate salt of the compound of formula (I), about 600 mg of the mesylate salt of the compound of formula (I), about 700 mg of the mesylate salt of the compound of formula (I), or about 800 mg of the mesylate salt of the compound of formula (I). In some preferred embodiments, the compound of formula (I) is administered at a dose equivalent to 300 mg of the mesylate salt of the compound of formula (I). In other preferred embodiments, the compound of formula (I) is administered at a dose equivalent to 450 mg of the mesylate salt of the compound of formula (I). In yet other preferred embodiments, the compound of formula (I) is administered at a dose equivalent to 500 mg of the mesylate salt of the compound of formula (I).

In yet other aspects, disclosed are pharmaceutical compositions comprising about 10 mg to about 1000 mg of a compound of formula (I):

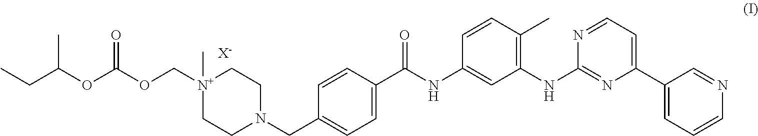

wherein X⁻ is a pharmaceutically acceptable anion;
and a pharmaceutically acceptable carrier or excipient; and uses thereof.

In certain especially preferred embodiments, X⁻ is mesylate. In some preferred embodiments, the composition is formulated for oral administration. In certain embodiments, the composition comprises about 10 mg of the compound of formula (I) to about 1000 mg of the compound of formula (I). In some embodiments, the composition comprises about 100 mg of the compound of formula (I), about 200 mg of the compound of formula (I), about 300 mg of the compound of formula (I), about 400 mg of the compound of formula (I), about 450 mg of the compound of formula (I), about 500 mg of the compound of formula (I), about 600 mg of the compound of formula (I), about 700 mg of the compound of formula (I), or about 800 mg of the compound of formula (I). In certain preferred embodiments, the composition comprises about 300 mg of the compound of formula (I). In other preferred embodiments, the composition comprises about 450 mg of the compound of formula (I). In yet other preferred embodiments, the composition comprises about 500 mg of the compound of formula (I).

In some embodiments, the composition comprises an amount of the compound of formula (I) equivalent to about 10 mg of the mesylate salt to about 1000 mg of the mesylate salt of the compound of formula (I). In certain embodiments, the composition comprises an amount of the compound of formula (I) equivalent to about 100 mg of the mesylate salt of the compound of formula (I), about 200 mg of the mesylate salt of the compound of formula (I), about 300 mg of the mesylate salt of the compound of formula (I), about 400 mg of the mesylate salt of the compound of formula (I), about 450 mg of the mesylate salt of the compound of formula (I), about 500 mg of the mesylate salt of the compound of formula (I), about 600 mg of the mesylate salt of the compound of formula (I), about 700 mg of the mesylate salt of the compound of formula (I), or about 800 mg of the mesylate salt of the compound of formula (I). In some preferred embodiments, the composition comprises an amount of the compound of formula (I) equivalent to 300 mg of the mesylate salt of the compound of formula (I). In other preferred embodiments, the composition comprises an amount of the compound of formula (I) equivalent to 450 mg of the mesylate salt of the compound of formula (I). In yet other preferred embodiments, the composition comprises an amount of the compound of formula (I) equivalent to 500 mg of the mesylate salt of the compound of formula (I).

In certain aspects, the present disclosure provides methods of treating pulmonary arterial hypertension, comprising administering to a subject a compound of Formula (II) or a pharmaceutically acceptable salt thereof:

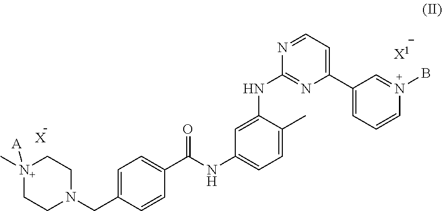

wherein:
A and B are independently selected from absent, H or a moiety of Formula (II), with the proviso that at least one of A and B is a moiety of Formula (III);

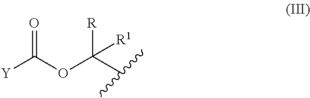

R and R¹ are each independently selected from H, alkenyl, alkynyl, alkoxy, aryl, heteroaryl, C₁-C₈ alkyl and C₃-C₇ cycloalkyl, wherein in each of the C₁-C₈ alkyl and C₃-C₇ cycloalkyl optionally up to three carbon atoms are replaced by a heteroatom group independently selected from O, NR⁴, S, SO and SO₂ (i.e., thereby making a heteroalkyl or heterocyclyl substituent), and wherein the C₁-C₈ alkyl and C₃-C₇ cycloalkyl are each optionally substituted with from 1 to 4 C₁-C₈ alkyl, alkoxy, aryl and heteroaryl substituents; or R and R¹ taken together with the atom to which they are attached form a 3- to 7-membered ring, wherein the 3- to 7-membered ring optionally contains up to two heteroatom groups selected from 0, N R⁴, S, SO and SO₂, and is optionally substituted with 1 to 4 alkoxy, F or Cl substituents;
Y is selected from R², OR², NH₂, NHR², and NR²R³;
R² is selected from alkoxy, aryl, heteroaryl, C₁-C₈ alkyl and C₃-C₇ cycloalkyl, wherein in each of the C₁-C₈ alkyl and C₃-C₇ cycloalkyl optionally up to three carbon atoms are replaced by a heteroatom group independently selected from O, NR⁴, S, SO and SO₂ (i.e., thereby making a heteroalkyl or heterocyclyl substituent), and wherein the C₁-C₈ alkyl and C₃-C₇ cycloalkyl are each optionally substituted with from 1 to 4 C₁-C₈alkyl, alkoxy, aryl or heteroaryl substituents;
R³ is selected from alkoxy, aryl, heteroaryl, C₁-C₈ alkyl and C₃-C₇ cycloalkyl, wherein in each of the C₁-C₈ alkyl and C₃-C₇ cycloalkyl optionally up to three carbon atoms are replaced by a heteroatom group independently selected from O, NR⁴, S, SO and SO₂ (i.e., thereby making a heteroalkyl or heterocyclyl substituent), and wherein the C₁-C₈ alkyl and C₃-C₇ cycloalkyl are each optionally substituted with from 1 to 4 C₁-C₈ alkyl, alkoxy, aryl or heteroaryl; or
R² and R³ may be taken together with the nitrogen atom to which they are attached to form a 3- to 7-membered ring, wherein the 3- to 7-membered ring optionally contains up to three heteroatom groups selected from O, NR⁴, S, SO and SO₂, and is optionally substituted with alkoxy, F or Cl;
R⁴ is, independently for each occurrence, selected from H or C₁-C₈ alkyl; and
X and X¹ are each independently an anion or absent, provided that X is absent only when A is absent, and X¹ is absent only when B is absent.

These compounds are described in U.S. Pat. No. 9,487,500, which is incorporated herein by reference in its entirety.

In some embodiments, R and R¹ are each independently selected from H and C₁-C₈ alkyl, such as H or methyl. Preferably both R and R¹ are H.

In some embodiments, R² and R³ are independently selected from C₁-C₈ alkyl and aralkyl. In some such embodiments, R² and R³ are independently selected from methyl, ethyl, isopropyl, tert-butyl, isobutyl, sec-butyl, 3-methylbut-2-yl, 1-phenylethyl, benzyl or cyclobutyl.

In some embodiments, R⁴, independently for each occurrence, is selected from H and C₁-C₈ alkyl.

In some embodiments, X and X¹ are each independently halide or sulfonate, such as mesylate and iodide.

Because anions are not covalently attached to the molecule, it should be understood that X and X¹ are not necessarily located proximal to the atom bearing A or B, and should be viewed as interchangeable within any given molecule when both are present.

In some embodiments, A is H and B is a moiety of Formula (III).

In some embodiments, A is a moiety of Formula (III) and B is H.

In other embodiments, A is a moiety of Formula (III) and B is absent.

In yet other embodiments, A is absent and B is a moiety of Formula (III).

In certain embodiments, neither A nor B is

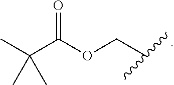 [SMILES: *COC(=O)C(C)(C)C]

The moieties of formula III may be divided into three classes: Type I, where Y═OR²; Type II, where Y═R² and Type III, where Y═NR²R³, wherein R² and R³ are as defined above. In some embodiments, formula III is selected from the moieties that would remain after displacing chlorine from the reagents listed below:

### Type I Reagents

i. chloromethyl isopropyl carbonate
ii. benzyl chloromethyl carbonate
iii. chloromethyl morpholinomethyl carbonate
iv. chloromethyl isobutyl carbonate
v. chloromethylmethyl carbonate
vi. (S)-sec-butyl chloromethyl carbonate
vii. (R)-sec-butyl chloromethyl carbonate
viii. chloromethyl ((3S,5R)-3,5-dimethylmorpholino)methyl carbonate
ix. chloromethyl 2-methylcyclopropyl carbonate
x. chloromethyl2-methoxyethyl carbonate
xi. chloromethyl propyl carbonate
xii. chloromethyl cyclobutyl carbonate
xiii. chloromethyl cyclopropyl carbonate
xiv. chloromethyl 2,2-dimethylcyclobutyl carbonate
xv. chloromethyl cyclopentyl carbonate
xvi. chloromethyl oxetan-3-yl carbonate
xvii. (S)-chloromethyl tetrahydrofuran-3-yl carbonate
xviii. chloromethyl cyclohexylmethyl carbonate
xix. chloromethyl 3-methoxycyclohexyl carbonate
xx. (R)-chloromethyl tetrahydrofuran-3-yl carbonate
xxi. chloromethyl ethoxymethyl carbonate
xxii. chloromethyl oxepan-4-yl carbonate
xxiii. (1R,2S,4S)-bicyclo[2.2.1]heptan-2-yl chloromethyl carbonate
xxiv. chloromethyl 2,3-dihydro-1H-inden-1-yl carbonate
xxv. benzyl chloromethyl carbonate
xxvi. (S)-chloromethyl 1-phenylethyl carbonate
xxvii. chloromethyl cyclohexyl carbonate
xxviii. chloromethyl isobutyl carbonate
xxix. chloromethyl 4-methylcyclohexyl carbonate
xxx. chloromethyl 2-(methylthio)ethyl carbonate
xxxi. chloromethyl 3-methylcyclohexyl carbonate
xxxii. chloromethylpentan-2-yl carbonate
xxxiii. chloromethyl neopentyl carbonate
xxxiv. methyl 1-((chloromethoxy)carbonyloxy)cyclopropanecarboxylate
xxxv. chloromethyl cyclopropylmethyl carbonate
xxxvi. chloromethyl 2,2-diethoxyethyl carbonate
xxxvii. chloromethyl cyclopentylmethyl carbonate
xxxviii. methyl 2-((chloromethoxy)carbonyloxy)propanoate
xxxix. (S)-chloromethyl 2,2,4-trimethylcyclopent-3-enyl carbonate
xl. chloromethyl 1,3-dioxolan-2-yl carbonate
xli. chloromethyl (2,6-dimethylcyclohexyl)methyl carbonate
xlii. chloromethyl 2-(tetrahydro-2H-pyran-2-yl)ethyl carbonate
xliii. chloromethyl(tetrahydro-2H-pyran-4-yl)methyl carbonate
xliv. chloromethyl tetrahydro-2H-pyran-4-yl carbonate
xlv. chloromethyl 1-methylcyclopentyl carbonate
xlvi. chloromethyl 1-cyclopentylethyl carbonate
xlvii. chloromethyl 3-methylcyclopentyl carbonate
xlviii. chloromethyl 3,3-dimethylcyclohexyl carbonate
xlix. chloromethyl 2,5-dimethylcyclohexyl carbonate
l. chloromethyl 1-(4-methylcyclohexyl)ethyl carbonate
li. chloromethyl (3-methyloxetan-3-yl)methyl carbonate
lii. chloromethyl (3-methyloxetan-3-yl)methyl carbonate
liii. chloromethyl 2-isopropoxyethyl carbonate
liv. (chloromethyl carbonic) 5-((3aS,4S,6aR)-2-oxohexahydro-1H-thieno[3,4-d]imidazol-4-yl)pentanoic anhydride
lv. 4-((chloromethoxy)carbonyloxy)-2-hydroxy-4-oxobutanoic acid
lvi. chloromethyl 4-formyl-2-methoxyphenyl carbonate
lvii. chloromethyl 3-oxobutan-2-yl carbonate
lviii. methyl 4-((chloromethoxy)carbonyloxy)benzoate
lix. (R)-2-amino-3-((chloromethoxy)carbonyloxy)propanoic acid
lx. 3-tert-butyl-4-methoxyphenyl chloromethyl carbonate
lxi. (R)-2-amino-3-(4-((chloromethoxy)carbonyloxy)phenyl)propanoic acid
lxii. (R)-2-amino-4-((chloromethoxy)carbonyloxy)-4-oxobutanoic acid
lxiii. (E)-chloromethyl 3,7-dimethylocta-2,6-dienyl carbonate
lxiv. methyl 4-((chloromethoxy)carbonyloxy)benzoate
lxv. chloromethyl 2-(4-methylcyclohex-3-enyl)propan-2-yl carbonate
lxvi. chloromethyl 3,7-dimethylocta-1,6-dien-3-yl carbonate
lxvii. 4-allyl-2-methoxyphenyl chloromethyl carbonate
lxviii. chloromethyl (1R,2S,5R)-2-isopropyl-5-methylcyclohexyl carbonate
lxix. propyl 4-((chloromethoxy)carbonyloxy)benzoate
lxx. (E)-chloromethyl 3,7-dimethylocta-2,6-dienyl carbonate

### Type II Reagents

i. chloromethyl cyclohexanecarboxylate
ii. chloromethyl 2-cyclohexylacetate
iii. chloromethyl 4-methylcyclohexanecarboxylate
iv. chloromethyl 1-methylcyclohexanecarboxylate
v. chloromethyl cyclopentanecarboxylate
vi. chloromethyl 1-(trifluoromethyl)cyclopentanecarboxylate
vii. chloromethyl cyclobutanecarboxylate
viii. chloromethyl 2-ethylhexanoate
ix. chloromethyl 3-cyclopentylpropanoate
x. chloromethyl cyclopropanecarboxylate
xi. chloromethyl pentanoate
xii. chloromethyl 2-methylpentanoate
xiii. chloromethyl 3,5,5-trimethylhexanoate
xiv. chloromethyl 2,2-dimethylbutanoate
xv. chloromethyl 2-methylbutanoate
xvi. chloromethyl hexanoate
xvii. chloromethyl 2-ethylbutanoate
xviii. chloromethyl butyrate
xix. chloromethyl 3-phenylpropanoate
xx. chloromethyl 2-phenylpropanoate
xxi. (R)-chloromethyl 2-phenylpropanoate
xxii. (S)-chloromethyl 2-phenylpropanoate
xxiii. (1r,4r)-chloromethyl 4-methylcyclohexanecarboxylate
xxiv. chloromethyl 4-methoxycyclohexanecarboxylate
xxv. chloromethyl 4,4-difluorocyclohexanecarboxylate
xxvi. chloromethyl 3-methoxycyclohexanecarboxylate
xxvii. (2R)-chloromethyl 2-methylcyclopentanecarboxylate
xxviii. (R)-chloromethyl 2-methylbutanoate
xxix. (S)-chloromethyl 2-methylbutanoate
xxx. (S)-chloromethyl 2-methoxy-2-phenylacetate
xxxi. (S)-chloromethyl 2-phenylpropanoate
xxxii. (S)-chloromethyl 2-phenylbutanoate
xxxiii. (S)-chloromethyl 3-phenylbutanoate
xxxiv. bis(chloromethyl) 2,2-dimethylmalonate
xxxv. bis(chloromethyl) oxalate
xxxvi. chloromethyl 2-cyclopropylacetate
xxxvii. chloromethyl 2-cyclobutylacetate
xxxviii. chloromethyl 2-cyclopentylacetate
xxxix. chloromethyl 2-(tetrahydrofuran-3-yl)acetate
xl. chloromethyl 2-(tetrahydro-2H-pyran-4-yl)acetate
xli. chloromethyl 2-methylcyclopropanecarboxylate
xlii. chloromethyl 2-(1-methylcyclobutyl)acetate
xliii. chloromethyl 2-(1-methylcyclopropyl)′acetate
xliv. chloromethyl propionate
xlv. chloromethyl acetate
xlvi. chloromethyl isobutyrate
xlvii. chloromethyl 2-isopropyl-3-methylbutanoate
xlviii. chloromethyl 3,5-dimethylcyclohexanecarboxylate
xlix. chloromethyl 2-propylpentanoate
l. chloromethyl 4-methoxybenzoate
li. chloromethyl 4-methylbenzoate
lii. chloromethyl 3-methylbenzoate
liii. chloromethyl 2,2,2-trifluoroacetate
liv. chloromethyl 5,5-dimethyl-3-oxohexanoate
lv. bis(chloromethyl) cyclopropane-1,1-dicarboxylate
lvi. chloromethyl 1,2-dihydrocyclobutabenzene-1-carboxylate
lvii. chloromethyl 2-cyclopentenylacetate
lviii. chloromethyl 2-phenylbutanoate
lix. chloromethyl 2,2-difluoroacetate
lx. chloromethyl 4-fluorobenzoate
lxi. chloromethyl 3-cyclohexylpropanoate
lxii. chloromethyl 2-cyclohexylacetate
lxiii. chloromethyl 3-(tetrahydro-2H-pyran-4-yl)propanoate
lxiv. chloromethyl 2-(tetrahydro-2H-pyran-3-yl)acetate
lxv. chloromethyl 3-(tetrahydro-2H-pyran-3-yl)propanoate
lxvi. chloromethyl nicotinate

### Type III Reagents

i. chloromethyl isopropylcarbamate
ii. chloromethyl diisopropylcarbamate
iii. chloromethyl dimethylcarbamate
iv. chloromethyl isobutylcarbamate
v. chloromethyl methylcarbamate
vi. chloromethyl ethyl(isopropyl)carbamate
vii. chloromethylisobutyl(methyl)carbamate
viii. (S)-chloromethyl sec-butylcarbamate
ix. chloromethyl methylcarbamate
x. chloromethyl isopropyl(methyl)carbamate
xi. chloromethyl propylcarbamate
xii. chloromethyl 2-methoxyethylcarbamate
xiii. chloromethyl methyl(propyl)carbamate
xiv. chloromethyl diisobutylcarbamate
xv. chloromethyl tert-butyl(isopropyl)carbamate
xvi. chloromethyl di-sec-butylcarbamate
xvii. chloromethyl aziridine-1-carboxylate
xviii. chloromethyl 2-methylcyclopropylcarbamate
xix. chloromethyl cyclopropylcarbamate
xx. chloromethyl cyclopropylmethyl(propyl)carbamate
xxi. chloromethyl cyclopropyl(methyl)carbamate
xxii. chloromethyl azetidine-1-carboxylate
xxiii. chloromethyl cyclobutylcarbamate
xxiv. chloromethyl 2,2-dimethylcyclobutylcarbamate
xxv. chloromethyl 3-methoxyazetidine-1-carboxylate
xxvi. chloromethyl cyclobutyl(methyl)carbamate
xxvii. chloromethyl oxetan-3-ylcarbamate
xxviii. (S)-chloromethyl 2-methylpyrrolidine-1-carboxylate
xxix. chloromethyl cyclopentylcarbamate
xxx. chlorometh1 cyclopentyl(methyl)carbamate
xxxi. chloromethyl tetrahydrofuran-3-ylcarbamate
xxxii. chloromethyl piperidine-1-carboxylate
xxxiii. (2R,6S)-chloromethyl 2,6-dimethylpiperidine-1-carboxylate
xxxiv. (R)-chloromethyl 2-methylpiperidine-1-carboxylate
xxxv. chloromethyl piperidine-1-carboxylate
xxxvi. chloromethyl 3-methoxycyclohexylcarbamate
xxxvii. chloromethyl cyclohexylmethylcarbamate
xxxviii. chloromethyl cyclohexylmethyl(methyl)carbamate
xxxix. chloromethyl morpholine-4-carboxylate
xl. (3S,5R)-chloromethyl 3,5-dimethylmorpholine-4-carboxylate
xli. (3R,5S)-chloromethyl 3,5-dimethylmorpholine-4-carboxylate
xlii. (2S,6R)-chloromethyl 2,6-dimethylmorpholine-4-carboxylate
xliii. chloromethyl 4-methylpiperazine-1-carboxylate
xliv. chloromethylazepane-1-carboxylate
xlv. chloromethylcycloheptylcarbamate
xlvi. chloromethyl oxepan-4-ylcarbamate
xlvii. chloromethyl (1R,2S,4S)-bicyclo[2.2.1]heptan-2-ylcarbamate
xlviii. chloromethyl 2,3-dihydro-1H-inden-1-ylcarbamate
xlix. chloromethyl benzylcarbamate
l. (S)-chloromethyl 1-phenylethylcarbamate
li. ethyl 2-((chloromethoxy)carbonylamino)-3-methylbutanoate
lii. ethyl 2-((chloromethoxy)carbonylamino)-3-phenylpropanoate
liii. (S)-diethyl 2-((chloromethoxy)carbonylamino)pentanedioate
liv. ethyl((chloromethoxy)carbonylamino)propanoate
lv. ethyl 2-amino-6-((chloromethoxy)carbonylamino)hexanoate
lvi. ethyl 2-((chloromethoxy)carbonylamino)-4-methylpentanoate
lvii. ethyl 2-((chloromethoxy)carbonylamino)-3-methylpentanoate
lviii. (S)-dimethyl 2-((chloromethoxy)carbonylamino)succinate
lix. (S)-ethyl 2-((chloromethoxy)carbonylamino)-5-guanidinopentanoate
lx. (S)-ethyl 4-amino-2-((chloromethoxy)carbonylamino)-4-oxobutanoate
lxi. (S)-ethyl 2-amino-5-((chloromethoxy)carbonylamino)pentanoate
lxii. (S)-ethyl 5-amino-2-((chloromethoxy)carbonylamino)-5-oxopentanoate
lxiii. ethyl 2-((chloromethoxy)carbonylamino)-4-(methylthio)butanoate
lxiv. 1-chloromethyl 3-methyl 2-methyl-5,6-dihydropyridine-1,3(2H)-dicarboxylate
lxv. (S)-chloromethyl (1-methylpyrrolidin-2-yl)methyl carbonate
lxvi. (R)-chloromethyl (1-methylpyrrolidin-2-yl)methyl carbonate
lxvii. (S)-(1-benzylpyrrolidin-2-yl)methyl chloromethyl carbonate
lxviii. chloromethyl 1H-pyrrole-1-carboxylate
lxix. chloromethyl 2-nicotinoylhydrazinecarboxylate
lxx. (6S)-3-chloro-7-((chloromethoxy)carbonylamino)-8-oxo-5-thia-1-azabicyclo[4.2.0]oct-2-ene-2-carboxylic acid
lxxi. (6S)-7-((chloromethoxy)carbonylamino)-8-oxo-3-vinyl-5-thia-1-azabicyclo[4.2.0]oct-2-ene-2-carboxylic acid
lxxii. (6S)-7-((chloromethoxy)carbonylamino)-3-(methoxymethyl)-8-oxo-5-thia-1-azabicyclo[4.2.0]oct-2-ene-2-carboxylic acid
lxxiii. (6R,7R)-7-((chloromethoxy)carbonylamino)-3-methoxy-8-oxo-5-thia-1-azabicyclo[4.2.0]oct-2-ene-2-carboxylic acid
lxxiv. chloromethyl 3-(4-chlorophenyl)-1H-pyrazole-1-carboxylate
lxxv. chloromethyl 3-(4-fluorophenyl)-1H-pyrazole-1-carboxylate
lxxvi. chloromethyl 3-phenyl-1H-pyrazole-1-carboxylate
lxxvii. chloromethyl 3-(4bromophenyl)-1H-pyrazole-1-carboxylate
lxxviii. chloromethyl 2-cyano-1H-pyrrole-1-carboxylate
lxxix. chloromethyl 4-oxopiperidine-1-carboxylate
lxxx. 1-chloromethyl 3-ethyl 2-oxopiperidine-1,3-dicarboxylate
lxxxi. chloromethyl 2,2,6,6-tetramethyl-4-oxopiperidine-1-carboxylate
lxxxii. chloromethyl 2-oxopiperidine-1-carboxylate

In some embodiments, the compounds of Formula (II) are selected from compounds of Formula (IV) or Formula (V):

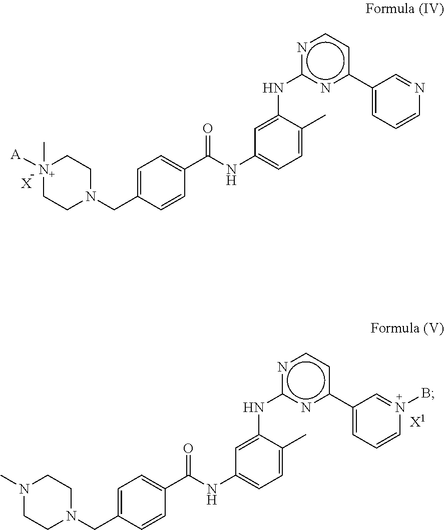

wherein A and B are independently selected from:

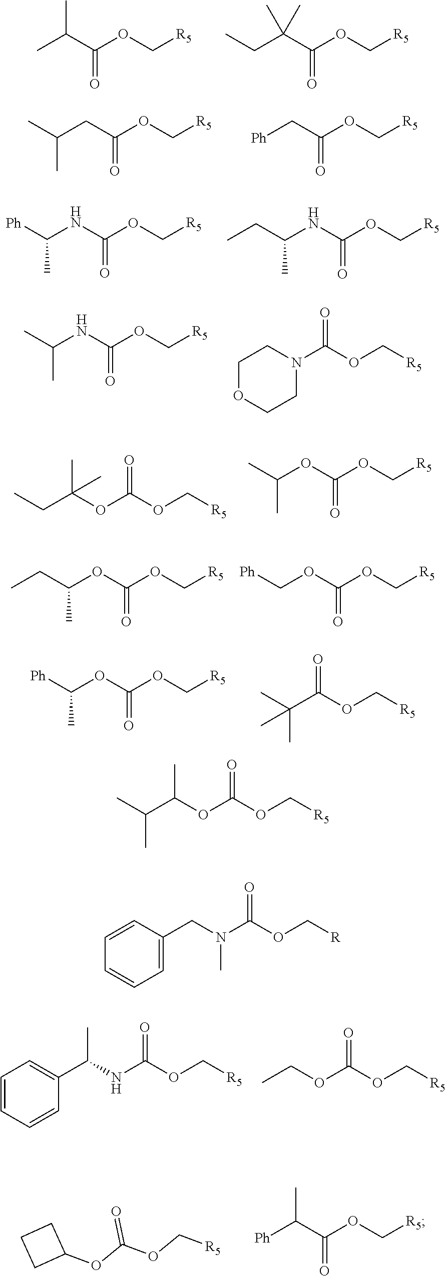

wherein R⁵ represents a nitrogen atom of the imatinib moiety linked to A or B, and
X may be iodide, chloride, bromide, mesylate, tosylate, or any other pharmaceutically acceptable anion to provide a pharmaceutically acceptable salt.

The compounds of Formula (II) may be present as a single stereoisomer (e.g., enriched to at least 95% purity relative to the total amount of all stereoisomers present), a racemate, or a mixture of enantiomers or diastereomers in any ratio.

In some embodiments, the compound of Formula (II) is selected from the compounds listed below:

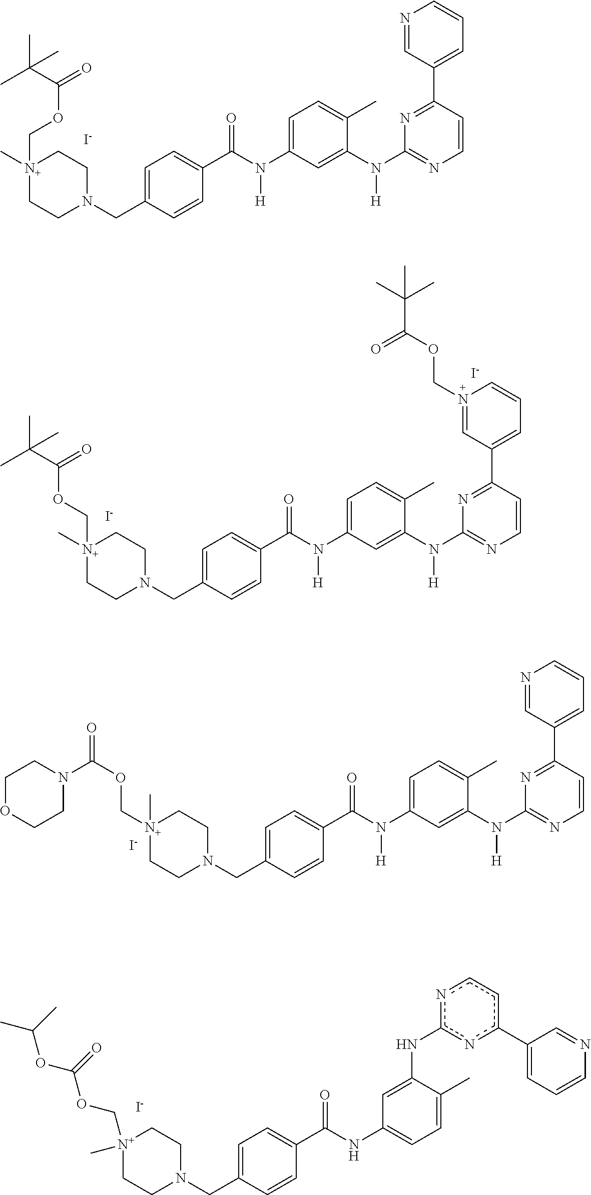

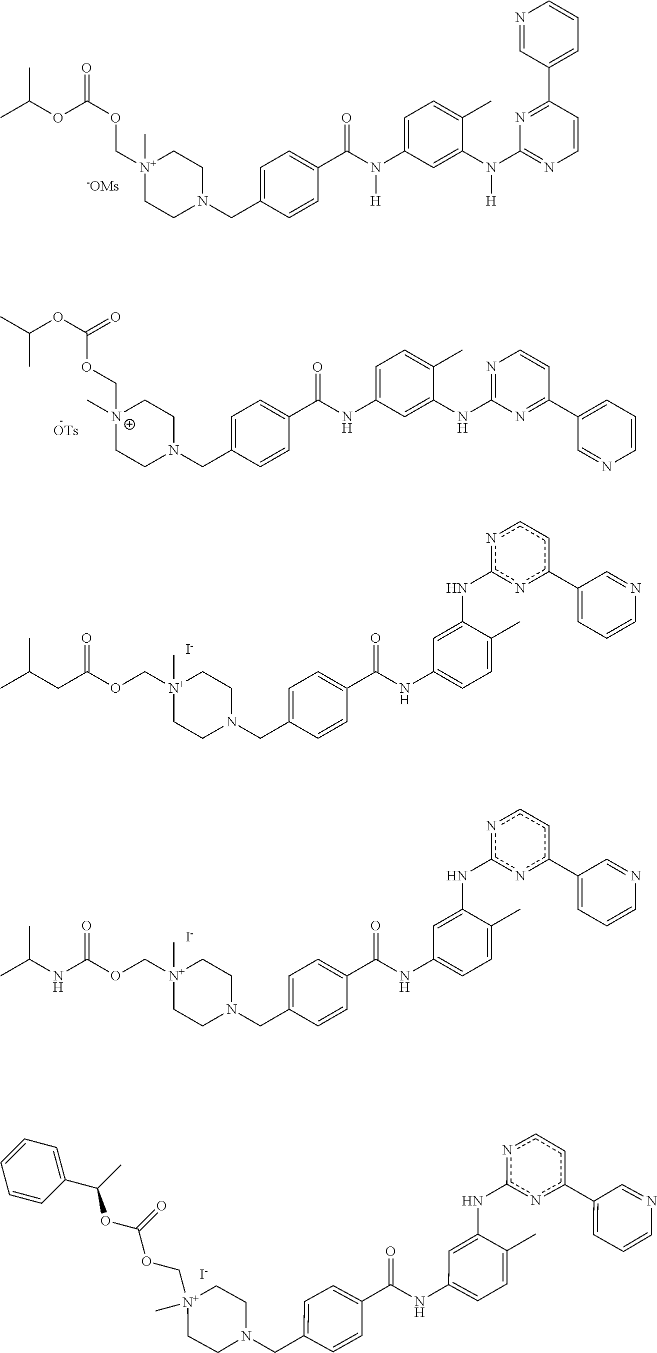

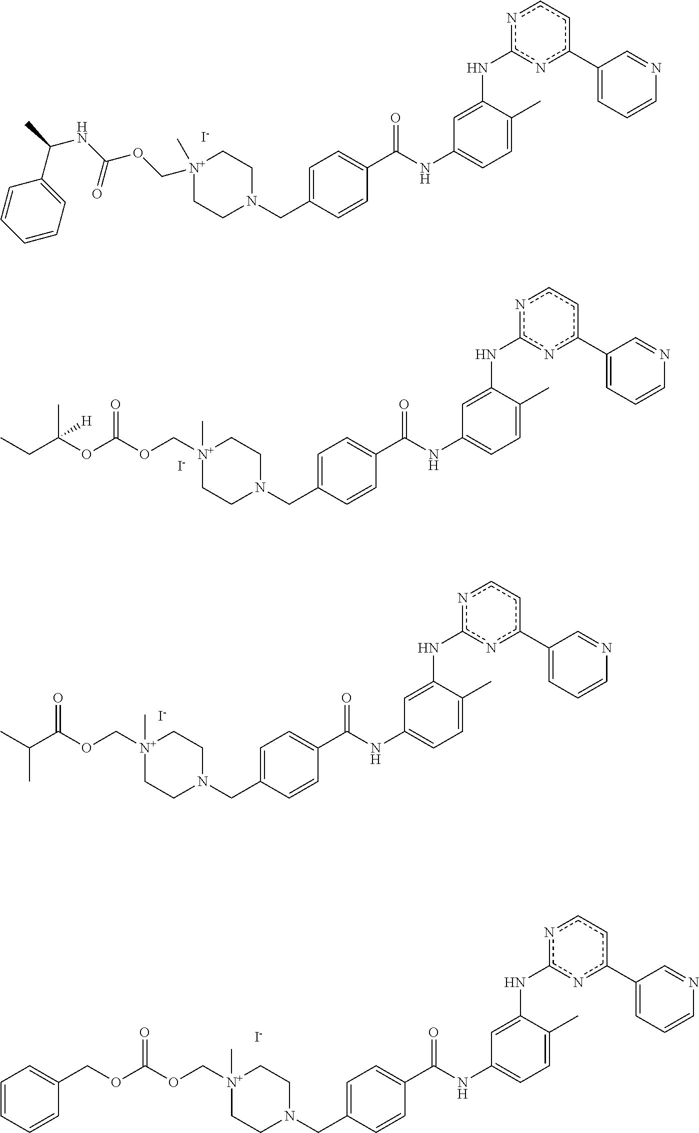

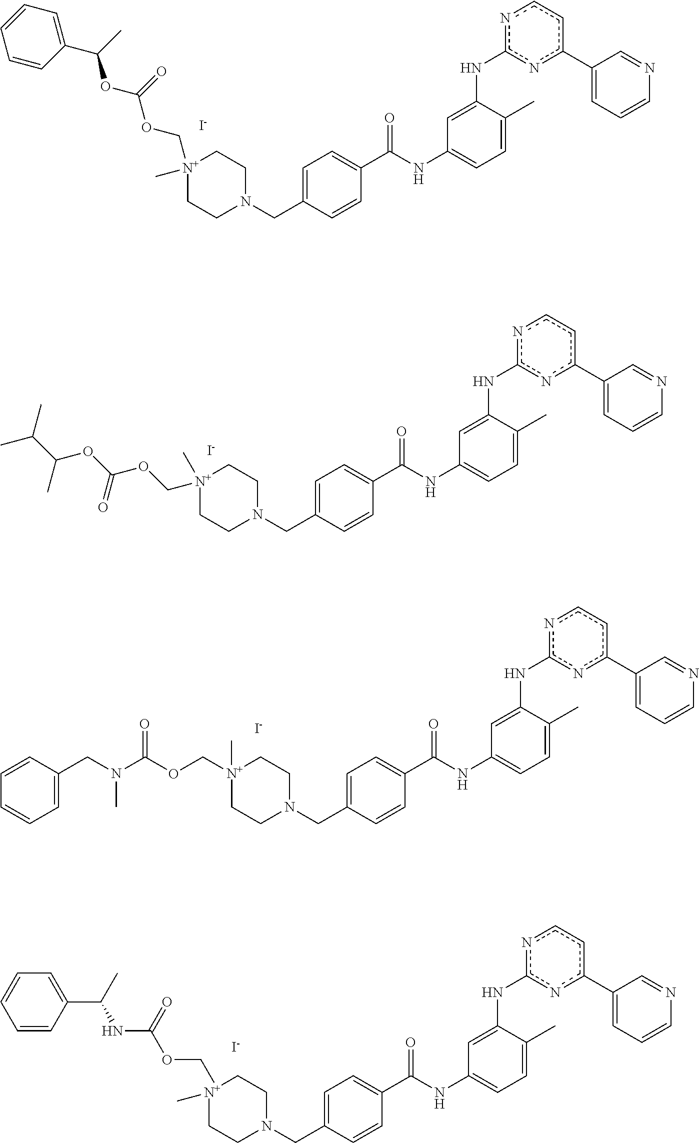

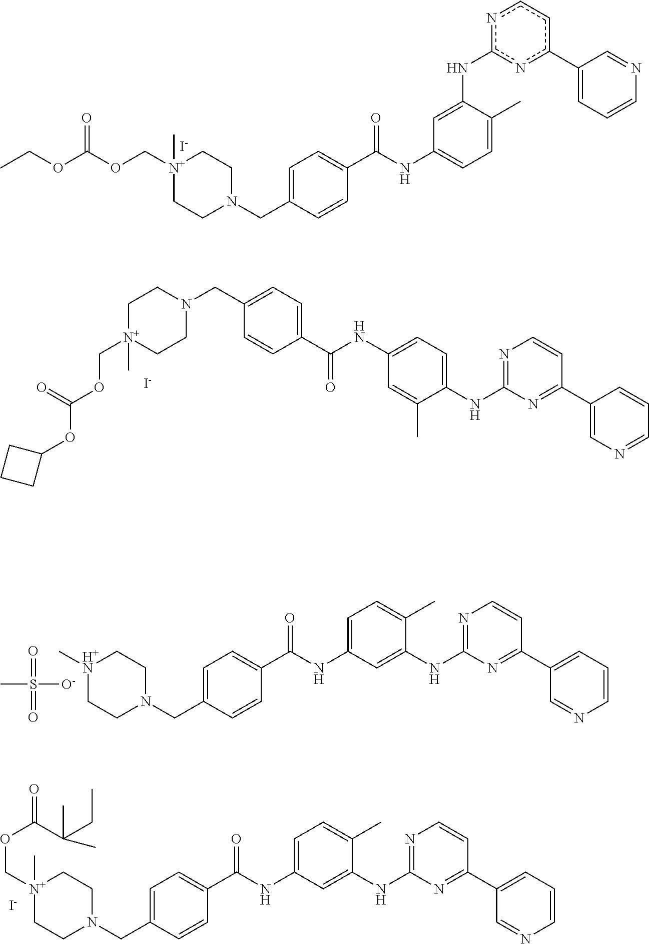

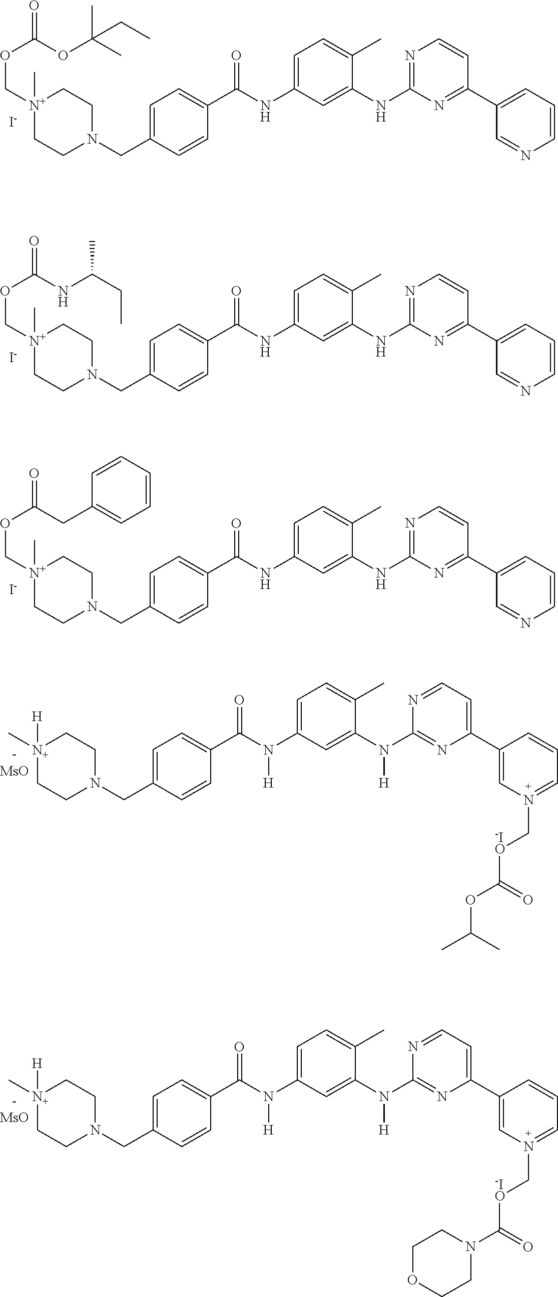

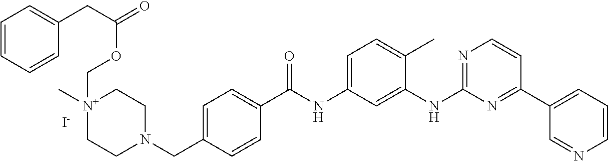 [SMILES: Cc1ccc(NC(=O)c2ccc(CN3CC[N+](C)(COC(=O)Cc4ccccc4)CC3)cc2)cc1Nc1nccc(-c2cccnc2)n1 ; [I-]]

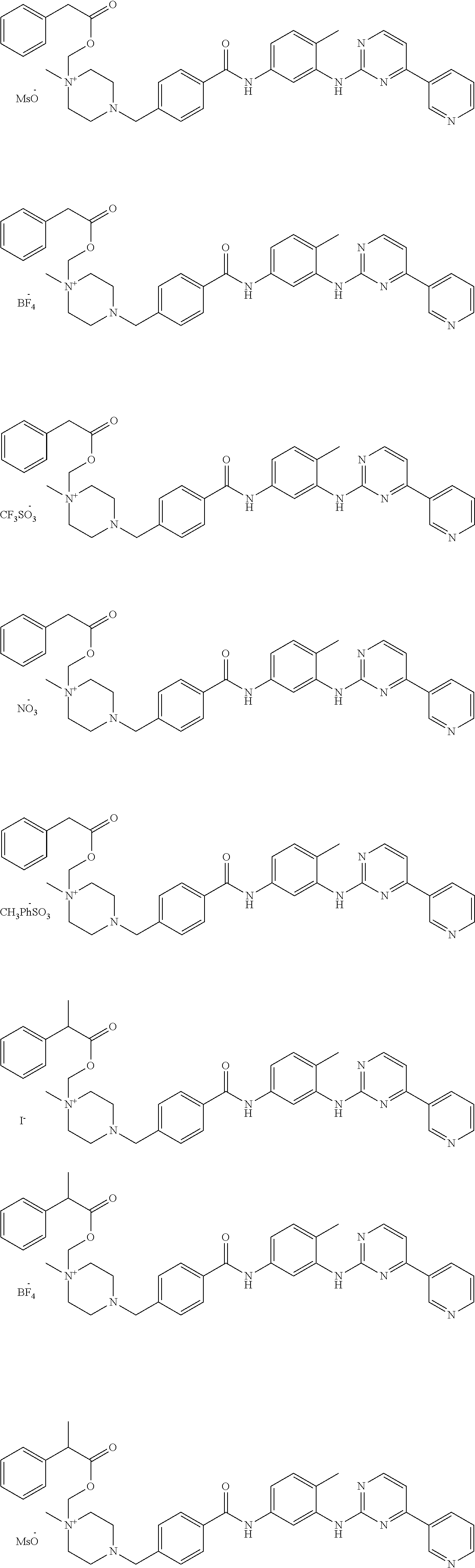

Pulmonary arterial hypertension (PAH) is an incurable and often fatal disease characterized by sustained elevation of pulmonary artery pressure and irreversible vascular remodeling. PAH can arise spontaneously, or can be caused by genetic mutations, drugs or environmental toxins. PAH is a rare condition that primarily afflicts women between the ages of 30 and 60 and can lead to premature heart failure and death. Previous clinical research with imatinib was shown to be potentially disease-modifying for PAH, however, the adverse event profile of imatinib mesylate in this patient population could not support approval by the FDA. Therefore, an imatinib prodrug may be a be a safer and better tolerated therapeutic option for imatinib treatment in PAH. PAH is also associated with connective tissue disease (CTD), congenital heart disease, HIV infection and other insults that could affect the right side of the heart. Most treatments for PAH attempt to address symptoms of this progressive disorder. There are approximately 30,000 cases of PAH in the U.S. The global PAH market size was valued at $7.66 billion in 2023 and is estimated to grow at a compound annual growth rate of 5.4% between 2024 to 2030.

Imatinib has been shown to have efficacy on par with other treatments for PAH, however its side effect profile precluded approval in this patient population. Changes in the standard-of-care for these patients suggests that the serious adverse events which arose from treatment with imatinib mesylate in the 2010s may not occur when imatinib is delivered as a prodrug. Despite the approval of several therapies treating PAH, there is currently no cure and patients continue to have a poor prognosis with a 5-year survival rate of 57%. Accordingly, improved agents for treating PAH are needed.

### Definitions

The term “alkyl” refers to the radical of saturated aliphatic groups, including straight-chain alkyl groups, and branched-chain alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In certain embodiments, alkyl groups are lower alkyl groups, e.g. methyl, ethyl, n-propyl, i-propyl, n-butyl and n-pentyl.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. In certain embodiments, a straight chain or branched chain alkyl has 30 or fewer carbon atoms in its backbone (e.g., C₁-C₃₀ for straight chains, C₃-C₃₀ for branched chains). In preferred embodiments, the chain has ten or fewer carbon (C₁-C₁₀) atoms in its backbone. In other embodiments, the chain has six or fewer carbon (C₁-C₆) atoms in its backbone.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. In preferred embodiments, a straight chain or branched chain alkenyl has 1-12 carbons in its backbone, preferably 1-8 carbons in its backbone, and more preferably 1-6 carbons in its backbone. Exemplary alkenyl groups include allyl, propenyl, butenyl, 2-methyl-2-butenyl, and the like.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated. In preferred embodiments, an alkynyl has 1-12 carbons in its backbone, preferably 1-8 carbons in its backbone, and more preferably 1-6 carbons in its backbone. Exemplary alkynyl groups include propynyl, butynyl, 3-methylpent-1-ynyl, and the like.

The term “aralkyl”, as used herein, refers to an alkyl group substituted with one or more aryl groups.

The term “aryl”, as used herein, include substituted or unsubstituted single-ring aromatic groups in which each atom of the ring is carbon. Preferably the ring is a 5- to 7-membered ring, more preferably a 6-membered ring. Aryl groups include phenyl, phenol, aniline, naphthyl, biphenyl, anthracenyl and the like.

The term “cycloalkyl”, as used herein, refers to the radical of a saturated aliphatic ring. In preferred embodiments, cycloalkyls have from 3-10 carbon atoms in their ring structure, and more preferably from 5-7 carbon atoms in the ring structure. Suitable cycloalkyls include cycloheptyl, cyclohexyl, cyclopentyl, cyclobutyl and cyclopropyl.

The terms “cycloalkyl” and “cycloalkenyl” refer to cyclic hydrocarbon groups of 3 to 12 carbon atoms.

The terms “halogen”, “halide” and “halo”, as used herein, mean halogen and include fluoro, chloro, bromo and iodo.

The terms “heterocyclyl”, “heterocycle”, “heterocyclo” and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The heterocyclic group may be attached at any heteroatom or carbon atom of the ring or ring system. Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, triazinyl, and the like. Exemplary bicyclic heterocyclic groups include indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo [2,3-c]pyridinyl, furo [3,2-b]pyridinyl] or furo [2,3-b]pyridinyl), dihydroisoindolyl, dihydroquinazolinyl (such as 3,4-dihydro-4-oxo-quinazolinyl), tetrahydroquinolinyl and the like. Exemplary tricyclic heterocyclic groups include carbazolyl, benzindolyl, phenanthrolinyl, acridinyl, phenanthridinyl, xanthenyl and the like.

The term “heteroalkyl”, as used herein, refers to a saturated or unsaturated chain of carbon atoms including at least one heteroatom (e.g., O, S, or NR⁴, such as where R⁴ is H or lower alkyl).

The term “heteroaryl” includes substituted or unsubstituted aromatic single ring structures, preferably 5- to 7-membered rings, more preferably 5- to 6-membered rings, whose ring structures include at least one heteroatom (e.g., O, N, or S), preferably one to four or one to 3 heteroatoms, more preferably one or two heteroatoms. When two or more heteroatoms are present in a heteroaryl ring, they may be the same or different. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Preferred polycyclic ring systems have two cyclic rings in which both of the rings are aromatic. Exemplary heteroaryl groups include pyrrolyl, pyrazolyl, imidazolyl, oxazolyl, isoxazolyl, thiazolyl, thiadiazolyl, isothiazolyl, furyl, thienyl, oxadiazolyl, pyridinyl, pyrazinyl, pyrimidinyl, quinolinyl, pyridazinyl, triazolyl, triazinyl, and the like.

The term “alkoxy” is intended to mean an alkyl radical, as defined herein, attached directly to an oxygen atom. Some embodiments are 1 to 5 carbons, some embodiments are 1 to 4 carbons, some embodiments are 1 to 3 carbons and some embodiments are 1 or 2 carbons. Examples include methoxy, ethoxy, n-propoxy, isopropoxy, n-butoxy, tert-butoxy, 5-isobutoxy, sec-butoxy, and the like.

The term “heteroatom”, as used herein, means an atom of any element other than carbon or hydrogen. Preferred heteroatoms are nitrogen, oxygen, and sulfur.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

Unless specifically stated as “unsubstituted,” references to chemical moieties herein are understood to include substituted variants. For example, reference to an “aryl” group or moiety implicitly includes both substituted and unsubstituted variants. Similarly, references to elements are understood to include any suitable isotope of that element. Thus, for example, a hydrogen substituent could be protium, deuterium, or tritium, or a carbon atom could be ¹²C, ¹³C, or ¹⁴C. In certain embodiments of the compounds disclosed herein, certain atoms may be isotopically enriched, e.g., for radioisotopic labelling or for a metabolically beneficial isotope effect (e.g., by isotopically enriching for deuterium at a hydrogen substituent). In such embodiments, the compound may be isotopically enriched for the desired isotope such that at least 15%, at least 25%, at least 50%, at least 60%, at least 75%, or even at least 90% more of the molecules of the compound in the composition have the desired isotope at the indicated position.

The term “unsaturated ring” includes partially unsaturated and aromatic rings.

As used herein, the term “tumoral disease” refers to a hyperproliferative disease, such as cancer.

As used herein, the term “conjoint administration” means administration of two or more agents to a subject of interest as part of a single therapeutic regimen. The administration(s) can be either simultaneous or sequential, i.e., administering one agent followed by administering of a second (and/or a third one, etc.) at a later time, as long as the agents administered co-exist in the subject being treated, or at least one agent will have the opportunity to act upon the same target tissues of other agents while said target tissues are still under the influence of said other agents. In a certain embodiment, agents to be administered can be included in a single pharmaceutical composition and administered together. In a certain embodiment, the agents are administered simultaneously, including through separate routes. In a certain embodiment, one or more agents are administered continuously, while other agents are administered only at predetermined intervals (such as a single large dosage, or twice a week at smaller dosages, etc.).

The present invention includes within its scope the salts and isomers. Compounds of the present invention may in some cases form salts, which are also within the scope of this invention. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Pharmaceutically acceptable (i.e., non-toxic, physiologically acceptable) salts are preferred, although other salts are useful, for example, in isolation or purification steps which may be employed during preparation. Salts of the compounds may be formed, for example, by reacting a compound with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxy ethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts (formed, for example, wherein the substituent comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the invention are also contemplated herein. Solvates of the compounds of formula I are preferably hydrates or other pharmaceutically acceptable solvates.

All stereoisomers of the present compounds, such as those which may exist due to asymmetric carbons on the R substituents of the compound, including enantiomeric and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention may have the S or R configuration.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

“Administering” or “administration of” a substance, or an agent to a subject can be carried out using one of a variety of methods known to those skilled in the art. For example, an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). An agent can also appropriately be introduced by rechargeable or biodegradable polymeric devices or other devices, e.g., patches and pumps, or formulations, which provide for the extended, slow or controlled release of the agent. Administering can also be performed, for example, once, a plurality of times, and/or over one or more extended periods.

Appropriate methods of administering a substance, or an agent to a subject will also depend, for example, on the age and/or the physical condition of the subject and the chemical and biological properties of the agent (e.g., solubility, digestibility, bioavailability, stability and toxicity). In certain embodiments, an agent is administered orally, e.g., to a subject by ingestion. In certain embodiments, the orally administered agent is in an extended release or slow release formulation, or administered using a device for such slow or extended release.

As used herein, the terms “mammal” or “subject” or “patient” includes both humans and non-humans and includes, but is not limited to, humans, non-human primates, canines, felines, mouse, bovine, equines, and porcines.

As used herein, the term “pulmonary arterial hypertension” refers to the condition characterized especially by increased or abnormally high blood pressure in the pulmonary artery, but also possibly in addition to other arteries or blood vessels of the pulmonary system. It will be understood to those of skill in the art how to appropriately determine whether a subject has and/or diagnose a subject with such a condition.

The present application also envisages within its scope the effect of selection of suitable counterions. The counterion of the compounds of the present invention may be chosen by selecting the dissociation constant for the drug capable of ionization within the said pH range.

By estimating the ionized and un-ionized drug concentration of any compound (using well established equations such a Henderson-Hasselbach equation), the solubility and consequently the absorption of the drug may be altered.

The compounds generated may be present as a single stereoisomer (e.g., enriched to at least 95% purity relative to the total amount of all stereoisomers present), a racemate, or a mixture of enantiomers or diastereomers in any ratio.

The term “prodrug” is intended to encompass compounds which, under physiologic conditions, are converted into the therapeutically active agents of the present invention (e.g., a compound of formula (I), such as its mesylate salt, which may interchangeably be referred to herein as IkT-001Pro). A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are preferred prodrugs of the present invention.

### Pharmaceutical Compositions

The present invention further provides pharmaceutical compositions comprising a compound of formula (I) or its pharmaceutically acceptable salt thereof as an active ingredient along with pharmaceutically acceptable additives/excipients/adjuvants/vehicles.

Compounds of the present invention may be used in a pharmaceutical composition, e.g., combined with a pharmaceutically acceptable carrier, for administration to a patient. Such a composition may also contain diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. Such additional factors and/or agents may be included in the pharmaceutical composition to produce a synergistic effect with compounds of the invention, or to minimize side effects caused by the compound of the invention.

The pharmaceutical compositions of the invention may be in the form of a liposome or micelles in which compounds of the present invention are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The composition may be administered in a variety of ways including orally, nasally, buccally, sublingually, intravenously, transmucosally, parenterally, by inhalation, spray, transdermally, subcutaneously, intrathecally, topically or rectally and may be formulated according to methods known in the art.

The effective dosage form for a mammal may be about 0.1-100 mg/kg of body weight of active compound, which may be administered as a single dose or in the form of individual doses, such as from 1 to 4 times a day. The mammal may preferably be an adult human.

The compounds of the present invention may optionally be administered with one or more additional agents. Exemplary additional agents include one or more compounds independently selected from central nervous system drugs, such as CNS/respiratory stimulants, analgesics, narcotic agonists, narcotic antagonists, nonsteroidal anti-inflammatory/analgesic agents, behavior-modifying agents, tranquilizers/sedatives, anesthetic agents, inhalants, narcotics, reversal agents, anticonvulsants, skeletal muscle relaxants, smooth muscle relaxants, cardiovascular agents, inotropic agents, antiarrhythmic drugs, anticholinergics, vasodilating agents, agents used in treatment of shock, alpha-adrenergic blocking agents, beta-adrenergic blocking agents, respiratory drugs, bronchodilators, sympathomimetics, antihistamines, antitussives, agents for urinary incontinence/retention, urinary alkalinizers, urinary acidifiers, cholinergic stimulants, agents for urolithiasis, gastrointestinal (GI) agents, antiemetic agents, antacids, histamine H2 antagonists, gastromucosal protectants, proton pump inhibitors, appetite stimulants, GI antispasmodics-anticholinergics, GI stimulants, laxatives, saline, bulk producing, lubricant, surfactant, antidiarrheals, hormones/endocrine/reproductive agents, sex hormones, anabolic steroids, posterior pituitary hormones, adrenal cortical steroids, glucocorticoids, antidiabetic agents, thyroid drugs, thyroid hormones, misc. endocrine/reproductive drugs, prostaglandins, antiinfective drugs, antiparasitics, anticoccidial agents, antibiotics, anti-tuberculosis, aminocyclitols, cephalosporins, macrolides, penicillins, tetracyclines, lincosamides, quinolones, sulfonamides, antibacterials, antifungal agents, antiviral agents, blood modifying agents, clotting agents, anticoagulants, erythropoietic agents, antineoplastics/immunosuppressives, alkylating agents, antidotes, bone/joint agents, dermatologic agents (systemic), vitamins and minerals/nutrients, systemic acidifiers, systemic alkalinizers, anti-cancer agents, and anti-viral agents.

### Methods of Use

The present invention further provides a method of prophylaxis and/or treatment of, and/or ameliorating the symptoms of, diseases, comprising administering a compound of formula (I), or pharmaceutical compositions comprising the compound of formula (I), as the active ingredient.

### Methods of Treating Pulmonary Arterial Hypertension

In certain aspects, the present invention provides methods of treating, inhibiting, or preventing pulmonary arterial hypertension (PAH) or its symptoms comprising administering to a subject in need thereof a compound of formula (I) as disclosed herein. In some embodiments, the methods comprise administering a pharmaceutical composition comprising a compound of formula (I) as described herein. In certain especially preferred embodiments, the compound of formula (I) is

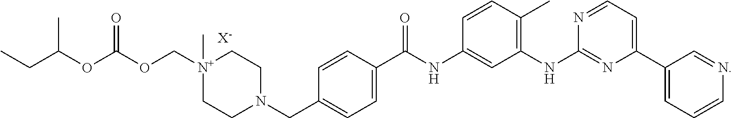

In certain preferred embodiments, the compound of formula (I) may be present as a pharmaceutically acceptable salt. In certain especially preferred embodiments, the compound of formula (I) is present as the mesylate salt. In these embodiments, 100 mg of the mesylate salt contains about 86.8 mg of the prodrug ion. The mass equivalence of other pharmaceutically acceptable counterions may be assessed according to this relationship.

The compound of formula (I) may be administered by any route known to those of skill in the art. In certain preferred embodiments, the compound of formula (I) or pharmaceutical composition is administered orally, nasally, buccally, sublingually, intravenously, transmucosally, parenterally, by inhalation, spray, transdermally, subcutaneously, topically or rectally. In certain preferred embodiments, the compound of formula (I) or pharmaceutical composition is administered orally or parenterally.

### EXAMPLES

The invention now being generally described, it will be more readily understood by reference to the following examples, which are included merely for purposes of illustration of certain aspects and embodiments of the present invention, and are not intended to limit the invention.

Exemplary Synthesis of Compounds of this Disclosure

The synthesis of the compounds of this disclosure have been reported previously in U.S. Patent Application No. PCT/US2013/063560, the contents of which are hereby incorporated by reference in their entirety.

### Example 1: Comparative Pharmacokinetics Studies

The compound of formula (I) was originally developed to address on-dosing side effects of imatinib therapy. In particular, up to 50% of imatinib patients experience persistent Grade 2 or less gastrointestinal (GI) adverse events during treatment for blood or stomach cancers, and during the previous imatinib trials, up to ⅓ of participants experienced these GI events including 3% of participants experiencing SAEs due to GI disturbance. The compound of formula (I) was developed specifically to reduce these events with the expectations that improvement in other off-target or unintended side effects of this therapy could follow. To illustrate the potential of the compound of formula (I) to minimize GI disturbance, the compound was evaluated in comparative toxicology experiments in non-human primate (NHP). The No Adverse Effect Level (NOAEL) for imatinib mesylate in NHP is just 15 mg/kg Q.D. However, the NOAEL of the compound of formula (I) is 75 mg/kg based on a 28-day GLP toxicology study in cynomolgus macaques. Comparison of the steady-state Cₘₐₓ and AUC_{0-inf} reveals the higher NOAEL of the compound of formula (I) increases the exposure to imatinib up to a 3.4-fold over imatinib mesylate, as demonstrated in Table 1.

TABLE 1
Steady-state Pharmacokinetics of IkT-001Pro
or imatinib mesylate in non-human primate.

| | No Adverse Event | Cₘₐₓ | | AUC₀₋ₗₐₛₜ |
|---|---|---|---|---|
| | Level (NOAEL) | (mean, | Tₘₐₓ | (mean, |
| | (mg/kg) | ng/mL) | (mean, h) | ng-h/mL) |
| Imatinib | 15 | 176/206 | 4/3 | 1540/1960 |
| (Day 91)¹ | | (M/F) | (M/F) | (M/F) |
| IkT-001Pro | 75 | 400/318 | 5.3/3.7 | 5220/3890 |
| (Day 28) | | (M/F) | (M/F) | (M/F) |

¹From NDA 21-335

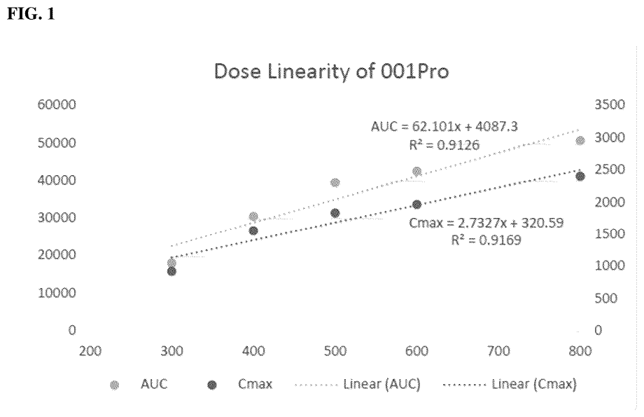

Additionally, the clinical pharmacokinetics for each cohort of healthy subjects studied for the compound of formula (I) using the designs were summarized. The single dose Cₘₐₓ and AUC_{0-inf} for the compound of formula (I) increased linearly with dose between 300 mg and 800 mg formula (I) freebase, although it was less than dose proportional (FIG. 1). The pharmacokinetic data supports the conclusion that 600 mg formula (I) delivers a bioequivalent dose of imatinib that is the same as 400 mg imatinib mesylate, as presented in Table 2.

TABLE 2
Statistical comparison of plasma imatinib pharmacokinetic
parameters following administration of 600 mg IkT-001Pro
compared to 400 mg commercial imatinib mesylate.
Table 2: Statistical Comparison of Plasma Imatinib
Pharmacokinetic Parameters Following Administration of 600 mg
IkT-001Pro Compared to 400 mg Commercial Imatinib Mesylate

| | | | Geo- | | | |
|---|---|---|---|---|---|---|
| | LSM | LSM | metric | | | Intra- |
| Param- | IkT- | Imatinib | Mean | Lower | Upper | subject |
| eters | 001Pro | Mesylate | Ratio | 90% CI | 90% CI | % CV |
| AUC_{0-inf} | 41202.597 | 35854.223 | 114.92 | 110.79 | 119.2 | 8.49 |
| AUC₀₋ₗₐₛₜ | 40456.384 | 35247.964 | 114.78 | 110.67 | 119.03 | 8.45 |
| Cₘₐₓ | 1939.51 | 1792.692 | 108.19 | 103.83 | 112.74 | 9.55 |

Parameters were ln-transformed prior to analysis
Treatment least-squares means (LSMEANS) are calculated by exponentiating the LSMEANS from the ANOVA.
Geometric Mean Ratio = 100*(600 mg 001Pro/400 mg imatinib mesylate)

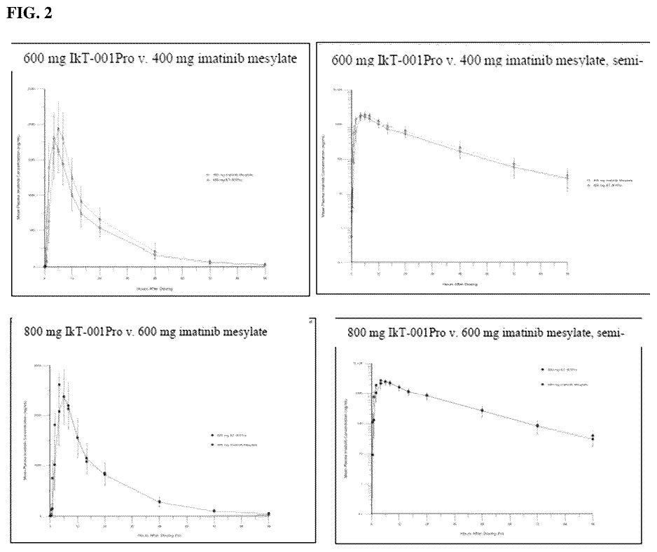

The pharmacokinetic data comparing 800 mg formula (I) to 600 mg imatinib mesylate (Table 3, FIG. 2), indicates that 800 mg formula (I) is just within the 80% to 125% limits of the 90% confidence interval and therefore could be the equivalent dose. For PAH, it is proposed to use much lower doses between 200 mg and 300 mg Q.D. of imatinib delivered. Given the dose linearity observed (FIG. 1), the corresponding doses of formula (I) are 300 mg and 450 mg, respectively.

TABLE 3
Statistical comparison following 800 mg IkT-
001Pro versus 600 mg imatinib mesylate.
Table 3: Statistical Comparison Following 800
mg IkT-001Pro Versus 600 mg Imatinib Mesylate

| | | | Geo- | | | |
|---|---|---|---|---|---|---|
| | IkT- | Imatinib | metric | | | Intra- |
| Param- | 001Pro | Mesylate | Mean | Lower | Upper | subject |
| eters | LSM | LSM | Ratio | 90% CI | 90% CI | % CV |
| AUC_{0-inf} | 50840.298 | 54493.269 | 93.30 | 81.28 | 107.09 | 14.63 |
| AUC₀₋ₗₐₛₜ | 50220.802 | 53634.103 | 93.64 | 81.58 | 107.47 | 14.63 |
| Cₘₐₓ | 2409.131 | 2637.178 | 91.35 | 78.66 | 106.09 | 15.89 |

Parameters were ln-transformed prior to analysis
Treatment least-squares means (LSMEANS) are calculated by exponentiating the LSMEANS from the ANOVA.
Geometric Mean Ratio = 100*(800 mg IkT-001Pro/600 mg Imatinib Mesylate)

TABLE 4
Single dose pharmacokinetics of imatinib released
from Formula (I) at 300, 400, 500, 600, and 800 mg.
Single dose pharmacokinetics of imatinib released
from IkT-001Pro at 300, 400, 500, 600 and 800 mg

| | | AUC₀₋ₗₐₛₜ | AUC_{0-inf} | Cₘₐₓ | Tₘₐₓ | t_{1/2} | CL/F | Vz/F |
|---|---|---|---|---|---|---|---|---|
| Treatment | | (hr*ng/mL) | (hr*ng/mL) | (ng/mL) | (hr) | (hr) | (L/hr) | (L) |
| 300 mg IkT- | N | 3 | 3 | 3 | 3 | 3 | 3 | 3 |
| 001Pro | Mean | 17820 | 18110 | 926.1 | 5.33 | 16.26 | 14.03 | 331.4 |
| Cohort 1 | SD | 5703.5 | 5778.8 | 259.35 | 1.16 | 0.72 | 4.51 | 119.22 |
| | CV % | 32 | 31.9 | 28 | 21.7 | 4.4 | 32.1 | 36.1 |
| 400 mg IkT- | N | 8 | 8 | 8 | 8 | 8 | 8 | 8 |
| 001Pro | Mean | 30100 | 30670 | 1563 | 5.75 | 15.93 | 10.82 | 247.5 |
| Cohort 2 | SD | 7615.6 | 7652.4 | 392.77 | 0.71 | 2.25 | 2.44 | 59.5 |
| | CV % | 25.3 | 25 | 25.1 | 12.3 | 14.1 | 22.5 | 24 |
| 500 mg IkT- | N | 8 | 8 | 8 | 8 | 8 | 8 | 8 |
| 001Pro | Mean | 38440 | 39620 | 1836 | 5.75 | 19.36 | 11.29 | 303.8 |
| Cohort 3 | SD | 12765 | 13466 | 613.4 | 0.71 | 3.61 | 4.68 | 92.94 |
| | CV % | 33.2 | 34 | 33.4 | 12.3 | 18.7 | 41.4 | 30.6 |
| 600 mg IkT- | N | 8 | 8 | 8 | 8 | 8 | 8 | 8 |
| 001Pro | Mean | 35610 | 36060 | 1839 | 6.25 | 16.01 | 13.61 | 316.4 |
| Cohort 4 | SD | 7375.1 | 7457.8 | 371.41 | 1.28 | 1.79 | 2.58 | 77.054 |
| | CV % | 20.7 | 20.7 | 20.2 | 20.5 | 11.2 | 19 | 24.4 |
| | N | 31 | 31 | 31 | 31 | 31 | 31 | 31 |
| 600 mg IkT- | Mean | 41830 | 42660 | 1974 | 5.935 | 15.98 | 11.88 | 268.7 |
| 001Pro | SD | 10862 | 11279 | 376.91 | 1.094 | 2.38 | 3.146 | 59.76 |
| Dose | CV % | 26 | 264 | 19.1 | 18.4 | 14.9 | 26.5 | 22.2 |
| Confirmation | | | | | | | | |
| Study | | | | | | | | |
| Cohort 5 | | | | | | | | |
| 800 mg IkT- | N | 8 | 8 | 8 | 8 | 8 | 8 | 8 |
| 001Pro | Mean | 51430 | 52090 | 2467 | 5.5 | 14.87 | 12.8 | 272 |
| Cohort 6 | SD | 11083 | 11335 | 548.76 | 1.41 | 1.38 | 3.61 | 67.44 |
| | CV % | 21.5 | 21.8 | 22.2 | 25.7 | 9.3 | 28.2 | 24.8 |

TABLE 5
Single dose pharmacokinetics of imatinib delivered by imatinib mesylate.
Single dose pharmacokinetics of imatinib delivered by imatinib mesylate

| | | AUC₀₋ₗₐₛₜ | AUC_{0-inf} | Cₘₐₓ | Tₘₐₓ | t_{1/2} | CL/F | Vz/F |
|---|---|---|---|---|---|---|---|---|
| Treatment | | (hr*ng/mL) | (hr*ng/mL) | (ng/mL) | (hr) | (hr) | (L/hr) | (L) |
| 400 mg | N | 8 | 8 | 8 | 8 | 8 | 8 | 8 |
| Imatinib | Mean | 43720 | 44540 | 2213 | 4 | 17.23 | 9.56 | 234.6 |
| mesylate | SD | 11572 | 11904 | 563.49 | 1.07 | 1.99 | 2.51 | 52.76 |
| Cohort 2 | CV % | 26.5 | 26.7 | 25.5 | 26.7 | 11.6 | 26.3 | 22.5 |
| 400 mg | N | 8 | 8 | 8 | 8 | 8 | 8 | 8 |
| Imatinib | Mean | 42880 | 44190 | 2102 | 4 | 18.74 | 10.1 | 265.2 |
| mesylate | SD | 14209 | 15079 | 582.5 | 0 | 4.01 | 3.83 | 82.14 |
| Cohort 3 | CV % | 33.1 | 34.1 | 27.7 | 0 | 21.4 | 37.9 | 31 |
| 400 mg | N | 8 | 8 | 8 | 8 | 8 | 8 | 8 |
| Imatinib | Mean | 31310 | 31760 | 1626 | 5 | 16.18 | 13.09 | 305.6 |
| mesylate | SD | 6942.8 | 7054.2 | 300.31 | 1.85 | 1.094 | 2.57 | 64.15 |
| Cohort 4 | CV % | 22.2 | 22.2 | 18.5 | 37 | 6.8 | 19.7 | 21 |
| 400 mg | N | 31 | 31 | 31 | 31 | 31 | 31 | 31 |
| Imatinib | Mean | 36150 | 36810 | 1823 | 4.065 | 15.99 | 11.49 | 260.6 |
| mesylate | SD | 8386.1 | 8735.3 | 354.67 | 0.81386 | 2.0786 | 2.8269 | 53.089 |
| Cohort 5 | CV % | 23.2 | 23.7 | 19.5 | 20 | 13 | 24.6 | 20.4 |
| 600 mg | N | 8 | 8 | 8 | 8 | 8 | 8 | 8 |
| imatinib | Mean | 54610 | 55530 | 2647 | 4.5 | 15.7 | 11.22 | 251.7 |
| mesylate | SD | 10991 | 11416 | 240.24 | 0.92582 | 2.1587 | 2.3585 | 53.664 |
| Cohort 6 | | | | | | | | |
| | CV % | 20.1 | 20.1 | 20.6 | 20.6 | 13.7 | 21 | 21.3 |

### Example 3: Clinical Study Design

This study will enroll approximately 150 participants at up to 50 sites globally. The study consists of two parts, a 26 week placebo controlled treatment period (Part A) followed by a 36 month extension period (Part B). Those participants that pass the screening process will be randomized during the baseline visit to either the low dose (300 mg) or placebo in a 2:1 ratio.

After two weeks participants will return to the clinic; upon confirmation that the participants are tolerating their dose, those that are on active treatment will be randomized to either 300 mg or 500 mg active treatment arms in a 1:1 manner. Those participants on placebo will remain on placebo for the rest of the 26-week placebo controlled treatment period. The final randomization structure for the 26-week placebo controlled treatment period will be such that participants are randomized in a 1:1:1 scheme to the 300 mg, 500 mg or placebo groups.

Participants who have not discontinued early will transition to a 36 month extension period. Participants who transition to the extension period will remain on the dose that they were assigned to after the two week acclimation period. Participants who were randomized to the placebo group will be re-randomized 1:1 to either the 300 mg or 500 mg IkT-001Pro treatment groups. The study will be unblinded and investigators will be given treatment assignments once the primary endpoint analysis is completed.

All clinical staff, study investigators, and participants are blinded to study assignments throughout the trial. Enrolled participants eat within 2 hours prior to reporting to the site on Day 1 (Baseline) and undergo protocol-mandated procedures. Meals should not include citrus fruits or juice and they should not be consumed within 6 hours on either side of dosing. Each participant self-administers the study intervention on-site at the first treatment visit and then at home, as per treatment assignment. At home, enrolled participants should eat a meal within one hour prior to dosing. The treatment period consists of outpatient visits at Weeks 4, 8, 12, 16, 20 and 24. A safety follow-up occurs at Week 26. Telephone contacts at Weeks 2, 6, 10, 14, 18 and 22 occur to review and document changes in concomitant medications, assess dosing compliance, and document and/or address any adverse events/serious adverse events. Efficacy based assessments (PVR, cardiac output, 6MWD, WHO FC, NT-proBNP level, and clinical worsening assessments) are completed at baseline as well as week 8, 16, 24, and 26. PAH-SYMPACT®, QoL assessment, are completed at baseline as week 12 and 24. Exploratory assessments (Borg CR10, EuroQol-5 Dimension-5 Level, and electrocardiogram) are completed at baseline as well as week 12 and 24.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method of treating pulmonary arterial hypertension, comprising administering to a subject a compound of formula (I): 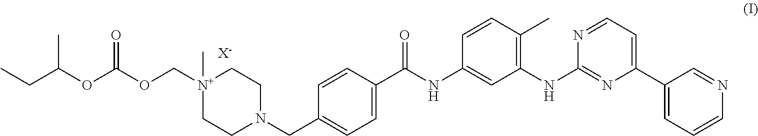
wherein X⁻ is a pharmaceutically acceptable anion.

2. The method of claim 1, wherein X⁻ is mesylate.

3. The method of claim 1, wherein the subject is a human.

4. The method of claim 2, comprising administering the compound at a dose of about 10 mg to about 1000 mg.

5. The method of claim 4, wherein the dose is about 100 mg per day, about 200 mg per day, about 300 mg per day, about 400 mg per day, about 450 mg per day, about 500 mg per day, about 600 mg per day, about 700 mg per day, or about 800 mg per day.

6. The method of claim 1, comprising repeatedly administering the compound at intervals (e.g., daily).

7. The method of claim 1, comprising administering the compound orally.

8. The method of claim 5, wherein the dose is 300 mg per day.

9. The method of claim 5, wherein the dose is 500 mg per day.

10. The method of claim 1, comprising administering the compound of formula (I) at a dose equivalent to about 10 mg of the mesylate salt of the compound of formula (I) to about 1000 mg of the mesylate salt of the compound of formula (I).

11. The method of claim 1, comprising administering the compound of formula (I) at a dose equivalent to about 100 mg of the mesylate salt of the compound of formula (I), about 200 mg of the mesylate salt of the compound of formula (I), about 300 mg of the mesylate salt of the compound of formula (I), about 400 mg of the mesylate salt of the compound of formula (I), about 450 mg of the mesylate salt of the compound of formula (I), about 500 mg of the mesylate salt of the compound of formula (I), about 600 mg of the mesylate salt of the compound of formula (I), about 700 mg of the mesylate salt of the compound of formula (I), or about 800 mg of the mesylate salt of the compound of formula (I).

12. The method of claim 1, comprising administering the compound of formula (I) at a dose equivalent to 300 mg of the mesylate salt of the compound of formula (I).

13. The method of claim 1, comprising administering the compound of formula (I) at a dose equivalent to 500 mg of the mesylate salt of the compound of formula (I).

14. A pharmaceutical composition comprising about 10 mg to about 1000 mg of a compound of formula (I): 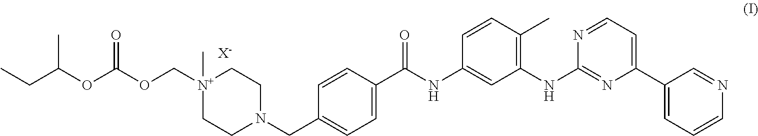
wherein X⁻ is a pharmaceutically acceptable anion;
and a pharmaceutically acceptable carrier or excipient.

15. The composition of claim 14, wherein X⁻ is mesylate.

16. The composition of claim 14, wherein the composition is formulated for oral administration.

17. The composition of claim 16, wherein the composition comprises about 10 mg of the compound of formula (I) to about 1000 mg of the compound of formula (I).

18. The composition of claim 15, wherein the composition comprises about 100 mg of the compound of formula (I), about 200 mg of the compound of formula (I), about 300 mg of the compound of formula (I), about 400 mg of the compound of formula (I), about 450 mg of the compound of formula (I), about 500 mg of the compound of formula (I), about 600 mg of the compound of formula (I), about 700 mg of the compound of formula (I), or about 800 mg of the compound of formula (I).

19. The composition of claim 15, wherein the composition comprises about 300 mg of the compound of formula (I).

20. The composition of claim 15, wherein the composition comprises about 500 mg of the compound of formula (I).

21. The composition of claim 14, wherein the composition comprises an amount of the compound of formula (I) equivalent to about 10 mg of the mesylate salt to about 1000 mg of the mesylate salt of the compound of formula (I).

22. The composition of claim 14, wherein the composition comprises an amount of the compound of formula (I) equivalent to about 100 mg of the mesylate salt of the compound of formula (I), about 200 mg of the mesylate salt of the compound of formula (I), about 300 mg of the mesylate salt of the compound of formula (I), about 400 mg of the mesylate salt of the compound of formula (I), about 450 mg of the mesylate salt of the compound of formula (I), about 500 mg of the mesylate salt of the compound of formula (I), about 600 mg of the mesylate salt of the compound of formula (I), about 700 mg of the mesylate salt of the compound of formula (I), or about 800 mg of the mesylate salt of the compound of formula (I).

23. The composition of claim 14, wherein the composition comprises an amount of the compound of formula (I) equivalent to 300 mg of the mesylate salt of the compound of formula (I).

24. The composition of claim 14, wherein the composition comprises an amount of the compound of formula (I) equivalent to 500 mg of the mesylate salt of the compound of formula (I).

25. A method of treating pulmonary arterial hypertension, comprising administering to a subject a compound of Formula (II) or a pharmaceutically acceptable salt thereof: 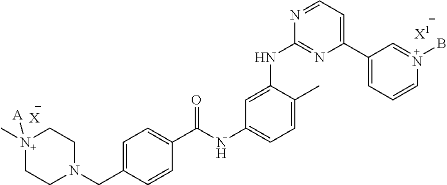
wherein:
A and B are independently selected from absent, H or a moiety of Formula (II), with the proviso that at least one of A and B is a moiety of Formula (III); 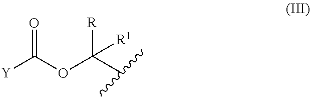
R and R¹ are each independently selected from H, alkenyl, alkynyl, alkoxy, aryl, heteroaryl, C₁-C₈ alkyl and C₃-C₇ cycloalkyl, wherein in each of the C₁-C₈ alkyl and C₃-C₇ cycloalkyl optionally up to three carbon atoms are replaced by a heteroatom group independently selected from O, NR⁴, S, SO and SO₂ (i.e., thereby making a heteroalkyl or heterocyclyl substituent), and wherein the C₁-C₈alkyl and C₃-C₇ cycloalkyl are each optionally substituted with from 1 to 4 C₁-C₈ alkyl, alkoxy, aryl and heteroaryl substituents; or R and R¹ taken together with the atom to which they are attached form a 3- to 7-membered ring, wherein the 3- to 7-membered ring optionally contains up to two heteroatom groups selected from O, N R⁴, S, SO and SO₂, and is optionally substituted with 1 to 4 alkoxy, F or Cl substituents;
Y is selected from R², OR², NH₂, NHR², and NR²R³;
R² is selected from alkoxy, aryl, heteroaryl, C₁-C₈ alkyl and C₃-C₇ cycloalkyl, wherein in each of the C₁-C₈ alkyl and C₃-C₇ cycloalkyl optionally up to three carbon atoms are replaced by a heteroatom group independently selected from O, NR⁴, S, SO and SO₂ (i.e., thereby making a heteroalkyl or heterocyclyl substituent), and wherein the C₁-C₈alkyl and C₃-C₇ cycloalkyl are each optionally substituted with from 1 to 4 C₁-C₈ alkyl, alkoxy, aryl or heteroaryl substituents;
R³ is selected from alkoxy, aryl, heteroaryl, C₁-C₈ alkyl and C₃-C₇ cycloalkyl, wherein in each of the C₁-C₈ alkyl and C₃-C₇ cycloalkyl optionally up to three carbon atoms are replaced by a heteroatom group independently selected from O, NR⁴, S, SO and SO₂ (i.e., thereby making a heteroalkyl or heterocyclyl substituent), and wherein the C₁-C₈alkyl and C₃-C₇ cycloalkyl are each optionally substituted with from 1 to 4 C₁-C₈ alkyl, alkoxy, aryl or heteroaryl; or
R² and R³ may be taken together with the nitrogen atom to which they are attached to form a 3- to 7-membered ring, wherein the 3- to 7-membered ring optionally contains up to three heteroatom groups selected from O, NR⁴, S, SO and SO₂, and is optionally substituted with alkoxy, F or Cl;
R⁴ is, independently for each occurrence, selected from H or C₁-C₈ alkyl; and
X and X¹ are each independently an anion or absent, provided that X is absent only when A is absent, and X¹ is absent only when B is absent.
